(19) United States
(12) Patent Application Publication
(10) Pub. No.: US 2011/0076781 A1
(43) Pub. Date: Mar. 31, 2011

(54) EXPANDING THE DYNAMIC RANGE OF A TEST STRIP

(75) Inventors: Hongjian Liu, Cupertino, CA (US); Robert K. DiNello, Rougemont, NC (US); Jimmy Z. Zhang, San Francisco, CA (US); George H. Sierra, El Paso, TX (US); Zhihai Ye, San Ramon, CA (US); Xiaohong He, Burlingame, CA (US); Zhibo Wu, Shenzhen (CN)

(73) Assignee: RELIA DIAGNOSTIC SYSTEMS, INC., San Francisco, CA (US)

(21) Appl. No.: 12/760,320

(22) Filed: Apr. 14, 2010

Related U.S. Application Data
(60) Provisional application No. 61/169,660, filed on Apr. 15, 2009.
(60) Provisional application No. 61/169,700, filed on Apr. 15, 2009.

(51) Int. Cl. G01N 33/566; G01N 33/00

(52) U.S. Cl. 436/501; 422/68.1

(57) ABSTRACT

The present invention provides a test strip with an expanded dynamic range for performing an assay to determine the presence of, or measure the quantity of, an analyte in a sample. It also provides methods for determining the presence of, or measuring the quantity of, an analyte in a sample, and for detecting a prozone sample.

This application claims the benefit of U.S. Provisional Application No. 61/169,660 entitled “Expanding the Dynamic Range of a Test Strip,” filed on Apr. 15, 2009, and of U.S. Provisional Application No. 61/169,700, entitled “Diagnostic Devices and Related Methods,” filed on Apr. 15, 2009, the disclosures of both of which are incorporated herein by reference in their entirety.

### BACKGROUND

Lateral flow assays are rapid tests which enable the user to detect analytes of interest in biological fluids in a point of care setting. However, current assays and test strips using this methodology are subject to a number of shortcomings which limit their usefulness. For example, the dynamic range is limited relative to assays using different methodologies including those carried out on large clinical analyzers. Samples need to be diluted so that concentrations in the physiologically relevant range may be measured by the lateral flow methodology.

Further, many of the current assays tend to exhibit a high dose hook effect (also known as prozone) in which very high concentrations of analyte may either give a reduced response or no response at all in a lateral flow assay. In such cases there is no way to distinguish the response from a high concentration sample from the response of a sample containing little or no analyte.

Thus there is a need for lateral flow assay methodologies with expanded dynamic ranges, that eliminate the necessity of diluting sample and which can detect samples in which a high dose hook effect has affected assay response.

### SUMMARY

The present invention is based, in part, on the discovery of a lateral flow assay methodology with an expanded dynamic range, that eliminates the need of diluting sample and which can detect prozone samples. Accordingly the present invention provides test strips for performing the assay and methods for determining the presence of, or measuring the quantity of, an analyte in a sample.

In one embodiment of the invention, it provides a test strip that comprises a chromatographic strip that comprises at least two reaction regions, a first reaction region, and a second reaction region, wherein each reaction region comprises a capture agent that specifically binds the same analyte that may be present in a sample, and wherein the two or more reaction regions expand the dynamic range of the test strip.

In another embodiment of the invention, it provides a test strip that comprises a chromatographic strip with a first end and a second end that comprises at least a first reaction region, and a second reaction region, wherein each reaction region comprises a capture agent that specifically binds the same analyte that may be present in a sample. The test strip further comprises an absorbent pad at the first end of the chromatographic strip and allows lateral flow of the sample such that as the sample is added to the chromatographic strip, the sample can flow across each reaction region, thereby allowing the capture agent therein to bind at least a part of the analyte present in the sample.

In yet another embodiment of the invention, it provides a method for detecting the presence of an analyte in a sample. The method comprises the steps of delivering a sample to a test strip of the invention; allowing the sample to flow along the test strip towards the reaction regions until it reaches the first reaction region and then the second reaction region; depleting the sample of analyte progressively by capturing at least a part of the analyte in the first reaction region, and if analyte remains in the sample, then in the second reaction region; determining the presence of the analyte in the sample based on intensity of a signal detected from the first reaction region, the second reaction region or a combination thereof; and optionally, measuring the quantity of, the analyte in the sample.

### BRIEF DESCRIPTION OF THE DRAWINGS

FIGS. 1-11 are depictions of different embodiments of the test strip of the invention.

FIG. 12 is a standard curve of relative intensity (RI) versus C-Reactive Protein (CRP) concentration from band Ti of a CRP assay described in Example 1.

FIG. 13 is a standard curve of RI versus CRP concentration from band T2 of the same CRP assay as in FIG. 12 showing that the standard curve for band T2 is displaced to higher CRP concentrations compared to the standard curve from band T1.

FIG. 14 is a standard curve of RI versus CRP concentration from the two band CRP assay of example 1 where the results of the two bands were combined by adding the relative intensities of each band showing an increased dynamic range.

FIG. 15 is a standard curve from band T1 of a CRP assay described in Example 2 (dynamic range is 0.162-5.412 mg/L).

FIG. 16 is a standard curve from band T2 of the same CRP assay as in FIG. 15 showing that the standard curve for band T2 is displaced to higher CRP concentrations compared to the standard curve from band T1 (dynamic range is 0.9-15 mg/L).

FIG. 17 is a standard curve from band T2 of the same CRP assay as in FIG. 15 showing that the standard curve for band T3 is displaced to higher CRP concentrations compared to the standard curve from band T2 (dynamic range is 6.6-20.44 mg/L).

FIG. 18 is a standard curve from a three band CRP assay where the results of all three bands (Bands T1 T2 and T3) were combined showing a further increase in dynamic range (dynamic range is 0.042-20.9 mg/L).

FIG. 19 is a standard curve from a three band CRP assay where the results of two of the three bands (Bands T2 and T3) were combined showing an increase in dynamic range (dynamic range is 0.25-19.58 mg/L).

FIG. 20 is a standard curve from a single test band NT-pro BNP assay with a dynamic range of from 85.65 pg/ml to about 3000 pg/ml.

FIG. 21 is a standard curve from a two test band NT-pro BNP assay with a dynamic range of from 88.89 pg/ml to about 15,000 pg/ml

### DETAILED DESCRIPTION

In one aspect, the present invention provides a test strip with an expanded dynamic range, that eliminates the need of diluting sample and which can detect prozone samples. In one embodiment, the test strip comprises a chromatographic strip that comprises at least two reaction regions, a first reaction region, and a second reaction region, wherein each reaction region comprises a capture agent that specifically binds an analyte that may be present in a sample, and wherein the two or more reaction regions expand the dynamic range of the test strip.

In another embodiment, the test strip comprises a chromatographic strip that comprises at least a first reaction region, and a second reaction region, wherein each reaction region comprises a capture agent that specifically binds the same analyte that may be present in a sample, wherein the presence of the analyte in the sample is to be determined. The test strip may, as described herein, comprise additional reaction regions, for example, a total of three, four, five, six, seven, eight, nine or ten reaction regions. The chromatographic strip has a first end and a second end and the test strip may further comprise an absorbent pad at the first end of the chromatographic strip.

The test strips of the invention may be dip sticks or test strips that support lateral flow, unidirectional or bi-directional lateral flow, of the sample such that when sample is added to the chromatographic strip it flows across each reaction region where the capture agent present therein binds at least a part of the analyte present in the sample.

The test strip may be configured such that the presence of the analyte in the sample is determined, or its concentration measured based on intensity of a signal detected from one or more of the reaction regions, e.g., the first reaction region, the second reaction region or a combination thereof

The term “dynamic range,” as used herein, for example, the dynamic range of a test strip is the range of concentrations over which the amount of analyte in a sample can accurately be determined by the test strip. The dynamic range of a test strip can be expanded such that the range of concentrations over which the amount of analyte in the sample can accurately be determined by the test strip is increased. In one embodiment, the range is expanded at the lower end of analyte concentration such that the test strip accurately determines lower concentrations of sample. In another embodiment, the range is expanded at the higher end of analyte concentration such that the test strip accurately determines higher concentrations of sample. In yet another embodiment, the range is expanded at both the lower and higher ends of analyte concentration.

The term “chromatographic strip” and “membrane strip” are used interchangeably herein and refer to a strip of any material that has sufficient porosity to allow the flow of fluid along its surface and through its interior. The fluid may flow due to capillary action, or any other means now known, or later discovered, for the flow of fluid along a membrane.

The term “first reaction region,” as used herein refers to the reaction region closest to the region on the chromatographic strip to which the sample is added. In one embodiment, the test strip comprises a sample addition zone to which the sample is added. The first reaction region would then be the reaction region closest to the sample addition zone such that, sample when added to the test strip flows along the chromatographic strip towards the reaction regions and reaches the first reaction region before reaching the second, third, fourth and subsequent reaction regions. The “second reaction region” is located a short distance away from, and in between the first reaction region and the third reaction region. Consistent with the use of first reaction region, second reaction region, etc., the “last reaction region” is located farthest from the sample addition zone and is reached last as the sample flows from the sample addition zone to the reaction regions.

As further described herein, the sample may be added to the first or the second end of the chromatographic strip. No matter to which end of the chromatographic strip the sample is added, the first reaction region is that reaction region closest to the region on the chromatographic strip to which sample is added/the sample addition zone.

The term “analyte,” as used herein refers to a compound that may be present in the sample and its presence and/or concentration in the sample are to be determined. An analyte may be any compound for which a specifically binding agent naturally exists or can be prepared. The term “analyte” further refers to both free/un-complexed analyte as well as to analyte that is bound by one or more analyte binding agents that may, or may not, be detectably labeled. Examples of analytes include, but are not limited to, proteins, such as hormones and other secreted proteins, enzymes, and cell surface proteins; glycoproteins; peptides; small molecules; polysaccharides; antibodies (including monoclonal or polyclonal antibodies); nucleic acids; drugs; toxins; viruses or virus particles; portions of a cell wall; and other compounds possessing epitopes.

The term “analyte binding agent,” as used herein refers to a moiety (or composition) that recognizes and binds the analyte. The term “capture agent” as used herein refers to a particular case of an analyte binding agent wherein the moiety (or composition) that recognizes and binds to the analyte is immobilized on the chromatographic strip such that when it binds the analyte, the analyte is “captured” on the test strip. Exemplary analyte binding agents include, but are not limited to, an antibody, an antigen, a peptide, a hapten, an engineered protein, nucleic acids, e.g., RNA, DNA, PNA, and other modified nucleic acids, as well as other biological and chemical molecules.

The term “antibody,” as used herein, includes an antibody binding region or fragment, one or more CDRs, single chain antibody, chimeric antibody, or humanized antibody. The antibody can be a monoclonal antibody or polyclonal antibody.

The sample can be any fluid sample, e.g., a biological sample such as a bodily fluid that is likely to contain the analyte of interest. In one embodiment, the biological sample is a blood, plasma, serum, saliva, mucus, urine, cervical mucus, or amniotic fluid sample. In another embodiment, the biological sample is a whole blood sample. In another embodiment, the sample is not a biological sample, but a fluid in which, for example, impurities or contaminants are to be detected. The sample may, but need not be treated prior to being deposited on the test strip. In certain cases where the sample is too viscous to flow evenly on the test strip, the sample may be pre-treated with agents that reduce the viscosity of the fluid, including, but not limited to, one or more mucolytic agents or mucinases.

In a preferred embodiment, pre-treatment of the sample does not include diluting the sample as the test strip of the invention has an expanded dynamic range, thereby removing the need to dilute the sample. In one embodiment, the dynamic range of the test strip of the invention can be up to 3, 4, 5, 6, or 7 logs. An exemplary dynamic range of the test strip of the invention includes, but is not limited to, about 0.0001 ng/ml to about 1 mg/ml. In one embodiment, the dynamic range of the test strip of the invention is from about 0.0001 ng/ml to about 750, 500, 400, 300, 200 100, 75, 50, 25, or about 10 μg/ml, or from about 0.0001 ng/ml to about 9, 8, 7, 6, 5, 4, 3, 2, or about 1 μg/ml. In another embodiment, the dynamic range of the test strip of the invention is from about 0.0001 ng/ml to about 750, 500, 400, 300, 200 100, 75, 50, 25, or about 10 ng/ml, or from about 0.0001 ng/ml to about 9, 8, 7, 6, 5, 4, 3, 2, or about 1 ng/ml, or from about 0.0001 ng/ml to about 0.5, 0.25, 0.1, 0.05, 0.025, 0.01, 0.005, or about 0.001 ng/ml. In yet another embodiment, the dynamic range of the test strip of the invention is from about 0.1, 0.2, 0.3, 0.4, 0.5, 0.6, 0.7, 0.8, 0.9, or about 1 μg/ml to about 1 mg/ml, or from about 1.5, 2, 2.5, 3, 4, 5, 7.5, 10, 15, 20, 25, 30, 35, 40, 45 or about 50 μg/ml to about 1 mg/ml. Intermediate ranges are also contemplated as part of the invention.

Based on the teachings of the invention, it would be within the scope of one of skill in the art to expand the dynamic range of the test strip as desired. For example, the dynamic range can be altered, i.e., expanded or reduced, by for example, adding or removing one or more reaction regions to the chromatographic strip. Alternatively, the dynamic range can be expanded or reduced, by changing the concentration of the capture agent present in the reaction region. Further, as taught herein, the accuracy and dynamic range of the test strip can be improved by varying the selection of the reaction region(s) from which signal is detected or measured.

The test strips of the invention may comprise just one capture agent, i.e., the same capture agent in the first, second and other, if any, reaction regions. Alternatively, they may comprise more than one, e.g., two, three, four, or more capture agents, that all bind the same analyte in the sample. In one embodiment, the test strip comprises two reaction regions, each of which comprises the same capture agent. In another embodiment, the test strip comprises two reaction regions comprising two different capture agents that both bind the same analyte. In yet another embodiment, the test strip of the invention comprises three or more reaction regions each of which comprises a capture agent that may, but need not, be identical to any of the other capture agents on the test strip.

Further, the two or more reaction regions of the test strip may comprise equal quantities or concentrations of capture agent, or the concentration may vary between the reaction regions. In general, the concentration of the capture agents in each reaction region can be adjusted to allow for an expanded dynamic range of the test strip.

In one embodiment, each reaction region of the test strip comprises the same capture agent present in the same quantity/concentration. In another embodiment, each reaction region of the test strip comprises the same capture agent, but the quantity or concentration of the capture agent may differ from region to region, e.g., the concentration in each region is different from any other region, or the concentration in say, two reaction regions is the same, but different from that in the third reaction region. In yet another embodiment, the reaction regions comprise different capture agents in either the same or different quantities or concentrations.

In an exemplary embodiment, the first reaction region, i.e., the reaction region nearest the sample addition zone, comprises a higher concentration of capture agent than the second, third and other reaction regions. This is particularly useful when the sample comprises a high concentration of the analyte, although it will work even when the analyte is present in low concentrations (in which case little or no signal is detected from the second reaction region and the third and further reaction regions, if present). In another embodiment, the first reaction region comprises a lower concentration of capture agent than the other reaction regions.

In some embodiments, the first or the last reaction region comprises the highest, the lowest, or an approximately even or equal concentration of capture agent compared to the other reaction regions. In one embodiment, the last reaction region comprises the highest or the lowest concentration of capture agent compared to the other reaction regions. In another embodiment, the first or the last reaction region comprises a concentration of capture agent that is in between those in the other reaction regions. Based on the teachings of this application, one of skill in the art will be able to vary the capture agent and/or the concentration of the capture agent based on the type and characteristics of the assay to be performed.

As stated above, the test strip may comprise several reaction regions, for example, three, four, five, six, seven, eight, nine, ten or more reaction regions. The capture agent present in each of the several reaction regions binds the same analyte in the sample, but need not be the same as the capture agent present in any of the other reaction regions.

The reaction regions can be of any shape or size, as determined by the kind of assay to be performed. In one embodiment, the reaction regions form a band, also referred to herein as a test band. In another embodiment, the reaction regions form a spot that can be of any shape, for example, approximately circular, oval or rectangular.

The test strips of the invention may further comprise features that enhance the ease of use, performance, sensitivity, accuracy or other characteristics of the test strips of the invention. For example, the test strip may comprise a sample addition zone that may, but need not, comprise a sample filter. The sample filter is capable of separating cells in the sample from fluid in the sample and allows the fluid to pass through to the chromatographic strip. This permits the use of samples such as whole blood to be used without pre-processing, as it allows the fluid to pass through to the chromatographic strip, but retains the cells.

The test strips of the invention may also comprise a conjugate region, optionally comprising a conjugate pad. The conjugate region or conjugate pad, if present, comprises a conjugate comprising an analyte binding agent labeled with a detectable marker. Fluid, e.g., sample or buffer, added to the chromatographic strip dissolves the conjugate and allows it to flow along the test strip. In one embodiment, the conjugate region/conjugate pad may be located on the chromatographic strip at or near the sample addition zone, or anywhere in between the sample addition zone and the first reaction region. When sample is added to the test strip, it dissolves the conjugate in the conjugate region/conjugate pad. Analyte in the sample binds the analyte binding agent in the conjugate and forms a detectable complex. This detectable complex continues to flow across the test strip until it reaches, and is captured in, one or more of the reaction regions.

In another embodiment, the test strip comprises both the sample filter and the conjugate pad. The sample filter may be located near the conjugate pad, or in one embodiment, located generally above the conjugate pad such that fluid in the sample that is added to the sample filter flows to the conjugate pad and from the conjugate pad onto and along the chromatographic strip. The sample filter may be directly over the conjugate pad, or alternatively, the sample filter may be offset, while still permitting fluid flow from the sample filter to the conjugate pad.

In yet another embodiment, the test strip comprises a buffer region, optionally comprising a buffer pad, to which buffer is added. When the test strip comprises a buffer region, the conjugate region or conjugate pad may be located on the chromatographic strip at or near the buffer region, or anywhere in between the buffer region and the reaction regions. When buffer is added to the buffer region or buffer pad it dissolves the conjugate and flows along the chromatographic strip carrying the conjugate to the reaction regions where conjugate can bind the immobilized analyte in the sample.

In a further embodiment, the test strip may comprise both the buffer pad and the conjugate pad. The buffer pad may be located near the conjugate pad, or in one embodiment, located generally above the conjugate pad such that fluid added to the buffer pad flows to the conjugate pad and from the conjugate pad onto and along the chromatographic strip. The buffer pad may be directly over the conjugate pad, or alternatively, the buffer pad may be offset, while still permitting fluid flow from the buffer pad to the conjugate pad.

In some embodiments, the conjugate is not present on the test strip, but is added to the sample before the sample is added to the test strip. Pre-mixing the conjugate that is present, for example, in a dried powder or liquid form, ensures that it is completely dissolved and increases the sensitivity of the assay.

In embodiments where the test strip comprises a conjugate, or when a conjugate is pre-mixed with the sample before the sample is added to the test strip, there are at least two agents that bind the analyte—one that is detectably labeled and one or more capture agents that are immobilized in the reaction regions. It is noted that at least one of the agents that bind the analyte should bind only to the analyte and not to any of the other components in the sample, i.e., should bind the analyte selectively or specifically. In one embodiment, the one or more capture agents that are immobilized in the reaction regions are analyte specific/selective and the analyte binding agent that is labeled with a detectable marker is capable of binding non-selectively to the analyte. In another embodiment, the one or more capture agents that are immobilized in the reaction regions are capable of binding non-selectively to the analyte and the analyte binding agent which is labeled with a detectable marker is analyte specific/selective. In yet another embodiment, both the capture agent(s) and the detectably labeled analyte binding agents are analyte specific/selective binding agents.

The detectable marker attached to the analyte binding agent may comprise a wide variety of materials now known, or later discovered, that permit the marker to be detected. Examples of detectable markers include, but are not limited to particles, luminescent labels; calorimetric labels, fluorescent labels; chemical labels; enzymes; radioactive labels; or radio frequency labels; metal colloids; and chemiluminescent labels.

Examples of common detection methodologies include, but are not limited to optical methods, such as measuring light scattering, simple reflectance, luminometer, photo diode or photomultiplier tube; radioactivity (measured with a Geiger counter, etc.); electrical conductivity or dielectric (capacitance); electrochemical detection of released electroactive agents, such as indium, bismuth, gallium or tellurium ions, as described by Hayes et al. (Analytical Chem. 66:1860-1865 (1994)) or ferrocyanide as suggested by Roberts and Durst (Analytical Chem. 67:482-491 (1995)) wherein ferrocyanide encapsulated within a liposome is released by addition of a drop of detergent at the detection zone with subsequent electrochemical detection of the released ferrocyanide. Other conventional methods may also be used, as appropriate.

It may be desired to assay two or more different analytes using the same test strip. In such instances, it may be desirable to employ different detectable markers on the same test strip where each detectable marker detects a different analyte. For example, different detectable markers may be attached to different analyte-selective binding agents. In one embodiment, the different detectable markers may be different fluorescent agents which fluoresce at different wavelengths.

In one embodiment, the detectable marker is a particle. Examples of particles that may be used include, but are not limited to, colloidal gold particles; colloidal sulphur particles; colloidal selenium particles; colloidal barium sulfate particles; colloidal iron sulfate particles; metal iodate particles; silver halide particles; silica particles; colloidal metal(hydrous)oxide particles; colloidal metal sulfide particles; colloidal lead selenide particles; colloidal cadmium selenide particles; colloidal metal phosphate particles; colloidal metal ferrite particles; any of the above-mentioned colloidal particles coated with organic or inorganic layers; protein or peptide molecules; liposomes; or organic polymer latex particles, such as polystyrene latex beads.

In one embodiment, the particles are colloidal gold particles. Colloidal gold particles may be made by any conventional method, such as the methods outlined in G. Frens, 1973 Nature Physical Science, 241:20 (1973). Alternative methods may be described in U.S. Pat. Nos. 5,578,577, 5,141,850; 4,775,636; 4,853,335; 4,859,612; 5,079,172; 5,202,267; 5,514,602; 5,616,467; and 5,681,775.

The selection of particle size may influence such factors as stability of bulk sol reagent and its conjugates, efficiency and completeness of release of particles from conjugate pad, speed and completeness of the reaction. Also, particle surface area may influence steric hindrance between bound moieties. Particle size may also be selected based on the porosity of the membrane strip. The particles are preferably sufficiently small to diffuse along the membrane with the fluid (sample or buffer).

Particles may be labeled to facilitate detection. Examples of labels include, but are not limited to, luminescent labels; colorimetric labels, such as dyes; fluorescent labels; or chemical labels, such as electroactive agents (e.g., ferrocyanide); enzymes; radioactive labels; or radio frequency labels.

The number of particles present in the test strip may vary, depending on the size and composition of the particles, the composition of the test strip and membrane strip, and the level of sensitivity of the assay. The number of particles typically ranges between about 1×10⁹ and about 1×10¹³ particles, although fewer than about 1×10⁹ particles may be used. In a preferred embodiment, the number of particles is about 1×10¹¹ particles.

In some embodiments, the test strip may comprise additional reaction regions that detect a control and which comprise one or more immobilized control binding agents. Controls or control agents capable of binding to the control binding agent may be positioned on the test strip at various locations or added to the test strip when the assay is being performed. The control agents may be labeled with a detectable marker, such as the detectable markers described above, to facilitate detection of the control when bound to the control binding agent immobilized in a control reaction region, also referred to herein as a “control zone” or a “control band.”

The control agents and control binding agents may be used in combination to perform a variety of control functions. For example, the control binding pairs may be used to confirm whether the sample or buffer have diffused properly within the test strip. The control binding pairs are also employable as internal standards and allow analyte measurement results to be compared between different test strips. This can be used to correct for strip-to-strip variability. Such correction would be impractical with external controls that are based, for example, on a statistical sampling of strips. Additionally, lot-to-lot and run-to-run variations between different test strips may be minimized by the use of control binding pairs. Furthermore, the effects of non-specific binding may be reduced. All of these corrections are difficult to accomplish using external, off-strip controls.

A wide variety of agents are known in the art which may be used as a member of the control binding pair. For example, at least one member of the control binding pair may be a naturally occurring or engineered protein. The control binding pair may also be a receptor-ligand pair. Additionally, at least one member of the control binding pair may be an antigen, another organic molecule, or a hapten conjugated to a protein non-specific for the analyte of interest. Descriptions of other suitable members of control binding pairs may be found in U.S. Pat. No. 5,096,837, and include IgG, other immunoglobulins, bovine serum albumin (BSA), other albumins, casein, and globulin.

Desirable characteristics for control agent—control binding agent pairs include, but are not limited to stability in bulk, non-specificity for analyte of interest, reproducibility and predictability of performance in test, molecular size, and avidity of binding for each other.

In one embodiment, members of the control binding pair do not bind to anything that might be present in the test strip, e.g., from the sample. In an exemplary embodiment, the control binding agent comprises rabbit anti-dinitrophenol (anti-DNP) antibody and the control agent includes a dinitrophenol conjugated to BSA (bovine serum albumin).

The test strips of the invention may also include a backing strip that runs the length of the test strip. The backing strip may be made of any stable, non-porous material that is sufficiently strong to support the materials and strips coupled to it. As many assays employ water as a diffusion medium, the backing strip is preferably substantially impervious to water. In one embodiment, the backing strip is made of a polymer film, more preferably a poly(vinyl chloride) film.

The chromatographic strip or membrane strip may be made of any substance that has sufficient porosity to allow the flow of fluid along its surface and through its interior. The fluid may flow due to capillary action, or any other means now known, or later discovered, for the flow of fluid along a membrane. The membrane strip should have sufficient porosity to allow movement of particles such as the conjugate. The membrane strip should also be wettable by the fluid used in the sample which contains the analyte to be detected (e.g., hydrophilicity for aqueous fluids, hydrophobicity for organic solvents). Hydrophobicity of a membrane can be altered to render the membrane hydrophilic for use with aqueous fluid, by processes such as those described in U.S. Pat. Nos. 4,340,482 or 4,618,533, which describe transformation of a hydrophobic surface into a hydrophilic surface. Examples of substances which can be used to form the membrane strip include: cellulose, nitrocellulose, cellulose acetate, glass fiber, nylon, polyelectrolyte ion exchange membrane, acrylic copolymer/nylon, and polyethersulfone. In one embodiment, the membrane is made of nitrocellulose.

The chromatographic strip may, but need not, be a single strip. In one embodiment, the chromatographic strip comprises a single, continuous strip. In another embodiment, the chromatographic strip comprises several smaller strips joined together to form a larger strip. The smaller strips may be shorter in length, but have approximately the same width as the larger strip, or the smaller strips may be narrower (smaller in width), but have approximately the same length as the larger strip, or the smaller strips may be both shorter and narrower than the larger strip. Further, the smaller strips may, but need not, comprise different regions of the chromatographic strip. In addition, the smaller strips may be placed adjacent to each other with no overlap, or they may overlap, partially or completely, with each other. Any arrangement of the smaller strips is possible as long as the larger strip thus formed allows the flow of fluid from its point of addition to the reaction regions.

The absorbent pad of the test strips of the invention may be formed of an absorbent substance that can absorb the fluid used as the sample and buffer. The absorption capacity of the absorbent pad should be sufficiently large to absorb the fluids that are delivered to the test strip. Examples of substances suitable for use in an absorbent pad include cellulose and glass fiber.

The buffer pad, conjugate pad and sample filter that are optionally present in the test strips of the invention may be formed of any absorbent substance. Examples of substances that may be used include cellulose, cellulose nitrate, cellulose acetate, glass fiber, nylon, polyelectrolyte ion exchange membrane, acrylic copolymer/nylon, and polyethersulfone.

The test strips of the invention may further comprise a protective cover that may be formed of any material which is impervious to water, and is preferably translucent or transparent. Preferable materials for use in the protective covering include optically transmissive materials such as polyamide, polyester, polyethylene, acrylic, glass, or similar materials. The protective covering may be clear or not clear depending on method of detection used. In one embodiment, protective covering is optically clear polyester.

In one embodiment, the test strip comprises a sample addition zone, located near the first end of a membrane strip, to which sample is added. The fluid sample when added to the test strip flows in both directions, i.e., towards the absorbent pad at the first end, and towards the second end, past the reaction regions. FIGS. 1-3 and 7-11 illustrate top-down views of exemplary embodiments of a lateral flow test strip according to the present invention. As illustrated, the test strip 2 has a membrane strip 4 with first and second ends 6 and 8, respectively. An absorbent region with an absorbent pad 10 is positioned at the first end of the membrane and the sample addition zone 12 is positioned near, but some distance away, from the first end. The first reaction region 14 and second reaction region 16 are positioned on the test strip between the sample addition zone and the second end 8. Additional reaction regions, e.g., a third reaction region may also be present on the test strip, as indicated by the dashed lines, and located between the second reaction region 16 and the second end 8. Each of the above mentioned regions or zones are in fluid diffusion communication with each other.

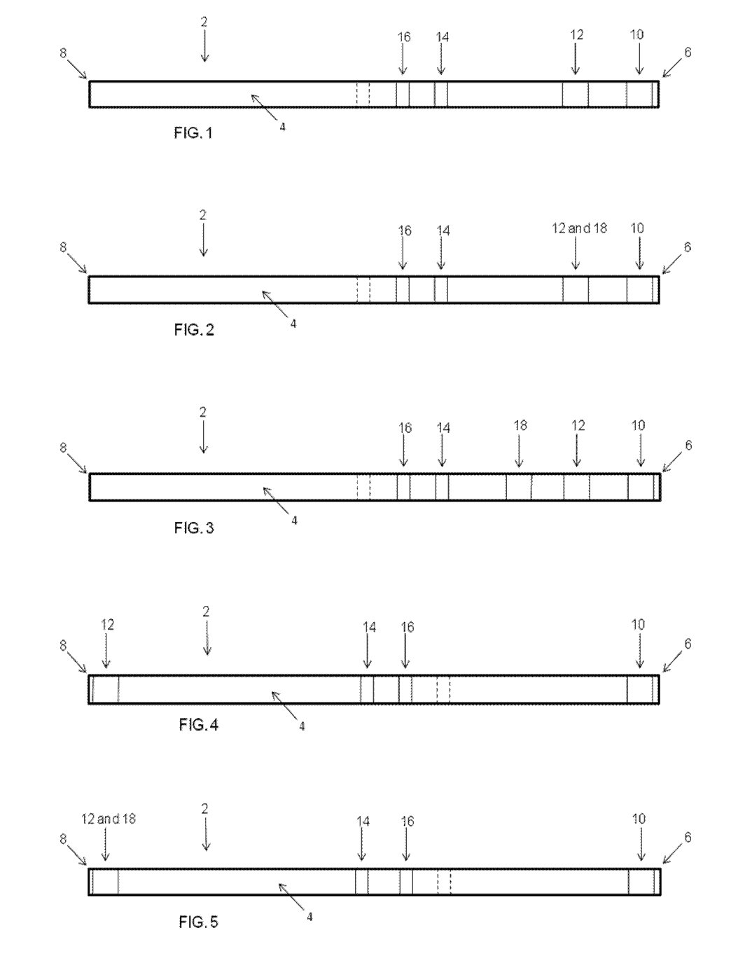

As depicted in FIGS. 2, 3, and 8-11, the test strip may further comprise a conjugate region and/or conjugate pad 18. In one embodiment, the conjugate region is, as illustrated in FIGS. 2 and 10, located at the sample addition zone, or as illustrated in FIGS. 3 and 11, located near the sample addition zone. In these embodiments, sample that is added to the test strip dissolves the conjugate and the analyte, if present in the sample, forms a detectable complex by binding the labeled analyte binding agent in the conjugate. This detectable complex flows along the test strip and is immobilized by binding the capture agent(s) in the reaction regions. This immobilized detectable complex can be detected or quantified. In the test strip configurations illustrated in FIGS. 10 and 11 the test strip further comprises a buffer region (that may, but need not, comprise a buffer pad) 20 that is located at the second end 8. Buffer added to the buffer region 20 flows towards the first end and washes excess analyte and/or conjugate off the reaction regions.

In another embodiment, as illustrated in FIGS. 8 and 9, the conjugate region/conjugate pad 18 is located at or near the second end of the membrane strip 8, respectively. In this embodiment, the sample is added at the sample addition zone 12 located near the first end of the membrane strip 6 and buffer is added to the buffer region/buffer pad 20. The sample flows along the test strip in both directions and analyte, if present in the sample is captured/immobilized by the capture agents in the first and subsequent reaction regions. The conjugate present at or near the second end of the membrane strip is dissolved by the fluid buffer and flows along the test strip until it reaches the reaction regions, where immobilized analyte binds the analyte binding agent in the conjugate to form an immobilized detectable complex that can be detected or quantified.

In the embodiments exemplified in FIGS. 1-3 and 7-11, the distance and/or the flow-rate between sample addition zone 12 and absorbent pad 10 and sample addition zone 12 and the reaction regions 14 and 16 can be adjusted such that sample does reach the absorbent pad before it has flowed past the first, second, and subsequent, if any, reaction regions.

In yet another embodiment, the sample addition zone is located at the second end of the membrane strip. The fluid sample, when added to the test strip, flows past the reaction regions, where analyte in the sample is captured by the capture agent(s), and reaches the absorbent pad at the first end. FIGS. 4-6 illustrate top-down views of exemplary embodiments of a lateral flow test strip according to the present invention. As illustrated, the test strip 2 has a membrane strip 4 with first and second ends 6 and 8, respectively. An absorbent region with an absorbent pad 10 is positioned at the first end of the membrane and the sample addition zone 12 is positioned at the second end. The first reaction region 14 and second reaction region 16 are positioned on the test strip between the sample addition zone and the absorbent pad 10. Additional reaction regions, e.g., a third reaction region may also be present on the test strip, as indicated by the dashed lines, and located between the second reaction region 16 and the absorbent pad 10. Each of the above mentioned regions or zones are in fluid diffusion communication with each other.

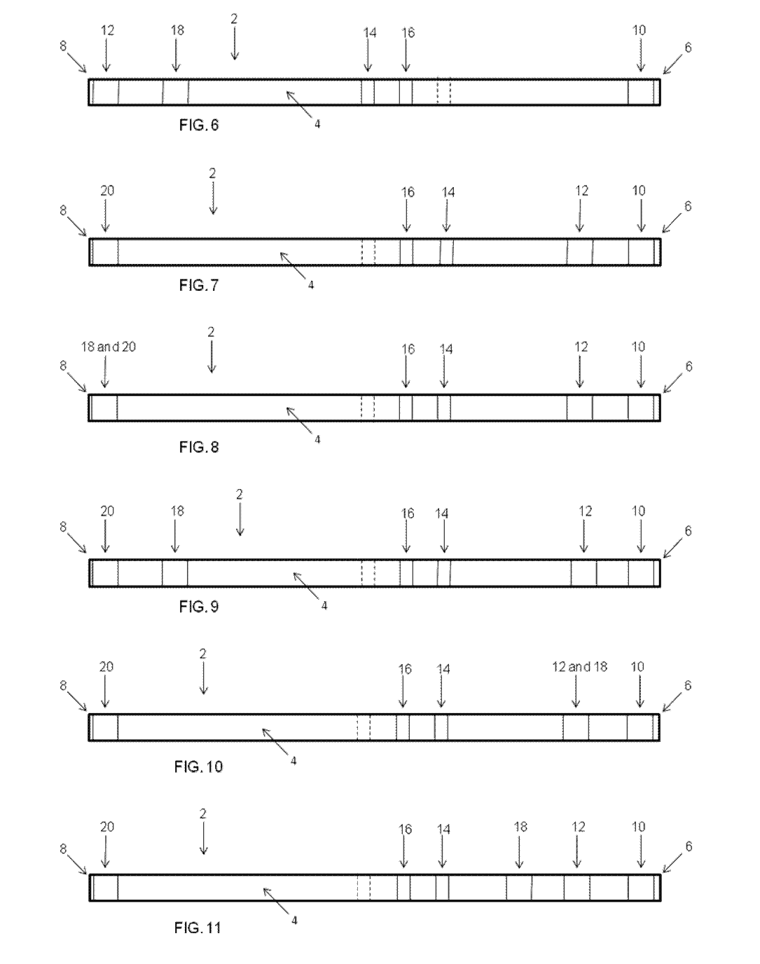

As illustrated in FIGS. 5 and 6, the test strip may further comprise a conjugate region and/or conjugate pad 18 that may be located at or near the sample addition zone, respectively. In these embodiments, sample that is added to the test strip dissolves the conjugate and the analyte, if present in the sample, forms a detectable complex by binding the labeled analyte binding agent in the conjugate. This detectable complex flows along the test strip and is immobilized by binding the capture agent(s) in the reaction regions. This immobilized detectable complex can be detected or quantified.

As illustrated in FIGS. 1-11, the test strips of the invention can be configured to allow unidirectional lateral flow or bi-directional lateral flow. Other configurations of the test strip that have not been illustrated are also considered part of the invention. For example, test strips of the invention may be configured such that the sample addition zone is located at the second end of the membrane strip and a buffer region/buffer pad is located near the first end.

Further, although the layout of the test strips illustrated in FIGS. 1-11 are linear in design, non-linear layouts are also contemplated as test strips according to the present invention. In one embodiment, the test strips of the invention is a “dip stick,” i.e., a strip wherein one end is dipped or placed in a fluid sample and the fluid sample flows along the strip. In another embodiment, the test strip of the invention is not a dip stick, but a lateral flow test strip.

In another aspect, the invention provides a method for determining the presence of an analyte in a sample. The method comprises the steps of delivering a sample to any test strip of the invention and allowing the sample to flow along the test strip towards the reaction regions until it reaches the first reaction region and then the second reaction region. The method further comprises progressively depleting the sample of analyte by capturing at least a part of the analyte in each reaction region. For example, in a test strip comprising two reaction regions, a first reaction region and a second reaction region, when the sample reaches the first reaction region at least a part of the analyte is captured by the capture agent in the first reaction region, thereby depleting the sample of analyte. When the sample reaches the second reaction region the sample is further depleted of analyte. The presence (or absence) of the analyte in the sample can be determined based on the presence (or absence) of a signal from the first reaction region, the second reaction region or a combination thereof, or from intensity of signal from the first reaction region, the second reaction region or a combination thereof. Optionally, the quantity or concentration of the analyte in the sample can be measured, from the intensity of the signal from the first reaction region, the second reaction region or a combination thereof.

When the test strip comprises three or more reaction regions the presence and or the quantity of analyte in the sample can be determined by the signal or signal intensity in any one of the reaction regions, or any combination thereof. In one embodiment, the signal or signal intensity from just one reaction region, for example the first reaction region or the last reaction region is determined. In another embodiment, signal or signal intensity from a combination of two or more reaction regions is determined.

The signals from any permutation of the reaction regions may be used for analyzing the results of the assay. For example, in an assay performed on a test strip with three test bands, each could be used separately for the low, middle and high range. Here the signal (and fitted curve) from the first reaction region is used to determine the value for samples in the lower range of concentrations. For samples with concentrations in the middle range the signal (and fitted curve) from the second reaction region will be used and for the upper range of concentration that from the third reaction region will be used.

In another embodiment, the signal (and fitted curve) from the first reaction region and a combination of the signals (and fitted curves) from the second and third reaction region can be used; i.e., T1 and T2+T3 are used (where T1, T2 and T3 represent the signal from the first, second and third test bands, respectively. In this embodiment, T1 is used for samples in the lower range of concentrations and the sum of the signals from the second and third test bands (T2+T3) is used for the remaining range of concentration.

In yet another embodiment, the sum of the signals from all three bands (i.e., T1+T2+T3) is used for the entire range of concentration. For assays that use only two bands, both methods (T1, T2) and (T1+T2) can be used.

In a further embodiment, when the first test band comprises a much lower concentration of the capture agent than the second test band which comprises a high concentration of the capture agent, then T2 can be used for the lower concentration and T1 for the mid and upper concentration ranges. Based on the teachings herein, the method to use can be determined empirically by one of skill in the art.

Methods of detecting the signal from a reaction region are known to one of skill in the art. The signal can be measured by using any detectable label that binds to the analyte. Examples of detectable labels or agents include, but are not limited to, luminescent agents, calorimetric agents such as dyes, fluorescent agents, chemical agents such as electroactive agents, radioactive agents, or radiofrequency agents. The selection of the detectable agents would depend on several factors including the size and composition of the agents, the composition of the chromatographic strip, the size of the reaction region, the level of sensitivity of the assay, etc. and is within the scope of one of ordinary skill in the art. Methods of measuring the intensity of a signal from a reaction region and determination of the quantity or concentration of an analyte in a sample are also known to one of skill in the art. Such measurements can, but need not, be performed with respect to a background or a baseline. Exemplary methods of measuring signal intensity and quantitative analysis of analyte concentration are taught, for example, in U.S. Pat. Nos. 6,528,323 and 6,767,710, each of which is incorporated herein by reference in its entirety.

In an exemplary embodiment, a test strip of the invention comprising one or two control bands in addition to two or three test bands is used to perform an assay. After completion of the assay, the test strip is illuminated using four white LEDs and a digital image of the strip is captured. The control and test bands are located and the peak density of reflection (DR) of each band is determined. If the DR that is measured in this step of the analysis were to be used to calculate a result, variations in the flow down the strip and other factors would cause poor reproducibility in results as measured with different strips. In order to normalize the results for these factors the DR of each test band is divided by the DR of the control band (if only one control band is present) or, when two control bands are present, then the DR of any one of the control bands or an average of the DR of the two control bands.

The capture agent(s) present in the control bands do not bind with any component of the sample and the intensity is simply a function of the concentration of the control coated on the chromatographic strip and the amount of conjugate on the conjugate pad. Under ideal conditions it would be expected that the intensity of the color developed from strip to strip on the control bands would be a constant. Variations that influence the measured intensity of the control bands will proportionately affect the test bands and the signals from the test bands can therefore be normalized using the value of one or both control bands. This normalized value is referred to herein as “relative intensity” (RI).

In order to determine the actual value of the analyte in the internationally recognized units corresponding to the measured RI, a response curve (also called a standard curve) must be determined. To make this determination, samples are manufactured with known values of the analyte over the entire range of the assay response. These are called standards. Using a statistically significant number of replicates for each standard that has been prepared, data is generated relating the RI developed to each standard value. A four parameter logistics curve fit is performed on this data. The resulting response curve (standard curve) can be used to convert the RI measured for each test band into the internationally recognized units for that analyte.

For assays where the result is basically either a negative or positive, e.g., an HIV assay, a signal to cut off (S/CO) is reported. As described above, the response of the assay is determined, except negative and positive clinical samples are used in place of standards. After statistically significant numbers of both positive and negative samples have been assayed, the mean value of the negative group and the associated standard deviation (SD) is calculated. An RI cut off value is determined that is a sufficient number of SDs away from the negative mean that a false positive is statistically unlikely, and is also less than the lowest RI value for a positive sample in the data. This cut off value is now used to calculate the S/CO for the results by dividing the assay RI by the cut off RI. Any result 1.0 or higher is a positive determination.

As discussed above, several data reduction/analysis methods can be used in the methods of the invention. In one embodiment, the test strip comprises two or three reaction regions or test bands and a standard curve is generated for each test band. In this embodiment, the method comprises using the standard curve for first test band until a preset or saturation value is exceeded. At that point, the method switches to using the standard curve for the second test band. In the case of three test bands, another switch point can be used at which point the standard curve for the third test band is used. In some embodiments, the total signal, e.g., total color developed in the test bands, is used. In these embodiments, a standard curve is created based on the total signal, e.g., color or relative intensity, and the presence and/or quantity of the analyte is determined from this curve by summing the total color of the two (or three) test bands. In some embodiments, a combination of a switch point from the first test band to reading the sum of the two test bands (or three bands) can be used.

In another embodiment, the test strip comprises two or three reaction regions, for example a first, second and optionally, a third test band, with the identical concentrations of capture agent, e.g., an antibody. The test strip also comprises a conjugate region comprising a conjugate that is dissolved by the sample and forms a detectable complex with the analyte. When the test strip is used in an assay, the first test band will ‘receive’ analyte-conjugate complex (hereinafter simply referred to as “analyte”) at 100% of full concentration. However, the succeeding test band (or test bands), i.e., the second and the third test band, will have attenuated levels of analyte with the amount of attenuation dependant on the concentration of the analyte present in the sample. At some point the first test band may saturate and the signal from the test band, e.g., color, may become a constant. High levels of analyte may even saturate two bands, the first and the second test bands, and the third test band may be used for assays where this quantity or concentration of analyte is present in the sample.

In yet another embodiment, the test strip comprises two or three test bands, wherein the first test band comprises a much lower concentration of the capture agent than the second test band which comprises a high concentration of the capture agent (i.e., the second test band will saturate at a low concentration of analyte). In this embodiment the dynamic range of the assay is extended as well as the sensitivity at the low end of the curve is improved. When analyte concentration in the sample is low, the first band has very little effect on the analyte concentration as very little binding occurs (due to a combination of the low concentration of capture agent in the first test band and the low concentration of the analyte). This barely-attenuated analyte then flows over the second test band (which is heavily coated with capture agent) and therefore with the proper selection of conjugate and capture agent concentrations the sensitivity at the low end of the analyte curve can be maximized. The second test band will saturate quickly and may even prozone. The detection and/or quantification of analyte in this embodiment can be performed by using the signal, e.g., color, of the second reaction region (second test band) for samples with low concentration of the analyte and switch to the signal, e.g., color, of the first reaction region (first test band) for samples with mid and high concentration of the analyte. In general, in this embodiment, the dynamic range is extended at the low-end (low level of analyte concentration) response.

Although the method of data reduction/analysis will vary from assay to assay, when the concentration of analyte in the sample is likely to be low, then the signal from the first test band is likely to be higher than that from the second test band, which, in turn is likely to be higher than that from the third test band. In this embodiment, the standard curve for first test band, or a standard curve created based on the total signal from the two (or three) test bands may be used. When the concentration of analyte in the sample is likely to be high (but not a prozone sample), the standard curve for third (or last) test band, or a standard curve created based on the total signal from the three test bands may be used.

In one embodiment, as the concentration of analyte in the sample increases, assay response from reaction regions closer to the sample addition zone reaches a maximum and then begins to decline while assay response from reaction regions farther from the sample addition zone continues to increase, resulting in a dose dependent increase in the ratio of response from reaction regions farther from the sample addition zone to response from reaction regions closer to the sample addition zone. As a consequence, a given response from a reaction region close to the sample addition zone may correspond to two different analyte concentrations on the dose-response curve, one low and one very high (prozone).

In one embodiment, a prozone sample can be detected by the following method. When the sample causes one or more test bands (e.g., the first test band or the first and second test band) to prozone then the standard curve for the next (e.g., second, or third, respectively) test band may be used. A prozone sample can be detected by comparing the signal from, for example, the first band and the second band, or the second band and the third band. If the signal is significantly lower in the first band as compared to the second band, or the second band as compared to the third band, then the sample is a prozone sample.

The standard curves (or individual response curves) for each reaction region may be combined to increase the range over which increased analyte dose results in increased test response.

### EXAMPLES

The following examples are intended to illustrate, but not to limit, the invention in any manner, shape, or form, either explicitly or implicitly. While they are typical of those that might be used, other procedures, methodologies, or techniques known to those skilled in the art may alternatively be used.

### Example 1

### Test Strip Preparation and Determination of CRP Concentration in a Sample

The test strips described in Thayer et al, U.S. Pat. No. 6,528,323 were prepared by coating Millipore HF 120 nitrocellulose (in order of distance from the sample application zone) High control (HC): 0.8 mg/ml rabbit anti-DNP, Test band 1 (T1): 0.5 mg/ml monoclonal anti-CRP 12D8, Test band 2 (T2): 0.5 mg/ml monoclonal anti-CRP 12D8, and low control (LC): 0.6 mg/ml rabbit anti-DNP. Antibodies were dissolved in PBS, 5% trehalose, 5% methanol for coating and the nitrocellulose was coated using an IVEK flatbed striper at 1 μl/cm. After coating, the HF 120 nitrocellulose was incubated overnight at 37° C. and then heat treated at 60° C. for four days.

Immunogold conjugates of monoclonal anti-CRP antibodies were prepared using 24 nm gold sol prepared by a modification of the method of Frens (Frens G., 1973. Controlled nucleation for the regulation of the particle size in monodisperse gold suspensions. Nature 241, 20-22) wherein a mixture of 160 ml 1% sodium citrate and 40 ml 1M sodium acetate were added to 8 L of boiling 18 megaohm deionized water. The solution was heated to boiling again and 80 ml of an aqueous solution of 1% gold chloride was added. After 13 minutes the solution turned bright red and after 20 minutes heating was discontinued and the solution was allowed to cool to room temperature. Conjugates were prepared by adding 1 ml of a mixture of 0.2 mg/ml of monoclonal anti-CRP 7D9 and 0.8 mg/ml nonspecific mouse IgG or 0.2 mg/ml ml of monoclonal anti-CRP 10C11 and 0.8 mg/ml nonspecific mouse IgG to 100 ml of the gold sol. After 10 minutes, 2 ml of 100 mg/ml Bovine Serum Albumin in 18 megaohm deionized water was added and the suspension stirred for 30 minutes at room temperature. The mixture was spun down at 13,000 RPM in a Beckman J2-21 centrifuge and the supernatant decanted from the red pellet containing the immunogold conjugate.

Immunogold conjugates of BSA-DNP (dinitrophenyl modified BSA were prepared in the following way: BSA-DNP was prepared by adding a 10 fold excess of DNP-X-SE (Invitrogen Molecular Probes) in dimethyl formamide to 10 ml of 10 mg/ml immunoglobulin free IgG in 1× PBS. After 1 hour at RT, the reaction mixture was spun down at 14,000 RPM in an Eppendorf Microfuge to remove yellow precipitate. The supernatant was concentrated to 1-1.5 ml and then chromatographed on Sephadex G-25 (Pharmacia) in 1×PBS to remove unreacted DNP-X-SE and its hydrolysis products to 100 ml of the gold sol. BSA-DNP immunogold conjugate of 28 nm gold was prepared by adding 1 ml of 1 mg/ml BSA-DNP to 100 ml of 28 nm gold sol. After 10 minutes, 2 ml of 100 mg/ml Bovine Serum Albumin in 18 megaohm deionized water was added and the suspension stirred for 30 minutes at room temperature. The mixture was spun down at 13,000 RPM in a Beckman J2-21 centrifuge and the supernatant decanted from the red pellet containing the immunogold conjugate. 28 nm gold sol was prepared as in b. (above) except 48 ml of 1% sodium citrate instead of 160 ml was used.

Conjugate pads (Millipore glass fiber) were prepared by mixing monoclonal monoclonal anti-CRP 7D9-24 nm gold conjugate (see b. above), monoclonal anti-CRP 10C11-24 nm gold conjugate (as described above) and BSA-DNP 28 nm gold (as described above). The mixture was diluted to the proper concentration with 2 mM borate, 0.1% PEG 20,000 and then mixed with an equal amount of 2 mM borate, 0.1% PEG 20,000, 10% trehalose and 1% casein. The contribution to the OD 520 in a 1:20 dilution of the final coating solution from monoclonal anti-CRP 7D9-24 nm gold conjugate (see b. above) was 0.65, from monoclonal anti-CRP 10C11-24 nm gold conjugate (see b. above) was 0.39 and from BDS-DNP-28 nm gold was 0.39. The conjugate mixture was striped on conjugate pads preblocked with 2 mM sodium borate pH 9, 0.1% PEG 20,000, 5% trehalose and 0.5% casein. Four lines were striped using a Biodot Quanti-3000 XYZ Dispensing Platform at 2.5 μl/cm. Conjugate pads were dried overnight under vacuum and then treated for four days at 60° C.

Sample pads were pre-blocked by dip coating Ahlstrom 141 pad material in: 0.6055% Tris, 0.12% EDTA.Na2, 1% BSA, 4% Tween 20 and 3.33% HBR-1. The material was dried at 37° C. for 2 hours and then vacuum dried overnight. Pre-blocked sample pads were cut into 10 mm wide strips using a G&L Drum Slitter.

Buffer pads were pre-blocked by dip coating Ahlstrom 141 pad material in: 0.6055% Tris, 0.12% EDTA.Na2, 1% BSA and 4% Tween 20. The material was dried at 37° C. for 2 hours and then vacuum dried overnight. Pre-blocked buffer pads were cut into 14 mm wide strips using a G&L Drum Slitter.

Test cards consisting of 70 mm×300 mm vinyl backing, coated 48 mm×300 mm nitrocellulose sheets, 10 mm×30 mm sample pad, 13 mm×300 mm conjugate pad and 14 mm×300 mm buffer pad were laminated together using a Kinematics Matrix Laminator and cut into 3.4 mm×70 mm strips. The strips were placed in cassettes described in Thayer et al. U.S. Pat. No. 6,528,323.

Assays using the strips described above were carried out in a ReLIA 2 Instrument (ReLIA Diagnostic Systems, Burlingame, Calif.). The cassette was placed in the cassette tray of the instrument and sample specific information is entered. A 50 μl sample of undiluted serum or plasma or a 60 μl sample of undiluted whole blood was added to Port 1 of the cassette described in Thayer et al. U.S. Pat. No. 6,528,323, initiating the assay sequence. The addition of sample was detected by a sensor and the cassette was withdrawn into the instrument for a countdown of 120 seconds. At the end of this time, the cassette tray was ejected from the instrument and the user was prompted to add 100 μl of conjugate release buffer (PBS 50 mg/ml BSA). Buffer addition was also detected by a sensor initiating the test sequence. The assay was carried out under predefined assay conditions (20 minutes at 33° C.). At the end of this time, the instrument determined the density of reflectance (DR) from each test and control band and the results were accessed using the computer interfaced with the instrument.

Standard samples of CRP were prepared by diluting a concentrated solution of human CRP into a human plasma pool containing little or no CRP. Results in this example were plotted as standard curves of RI (relative intensity, defined as the density of reflectance of the Test band (either T1 or T2) divided by the mean density of reflectance of the high and low controls.

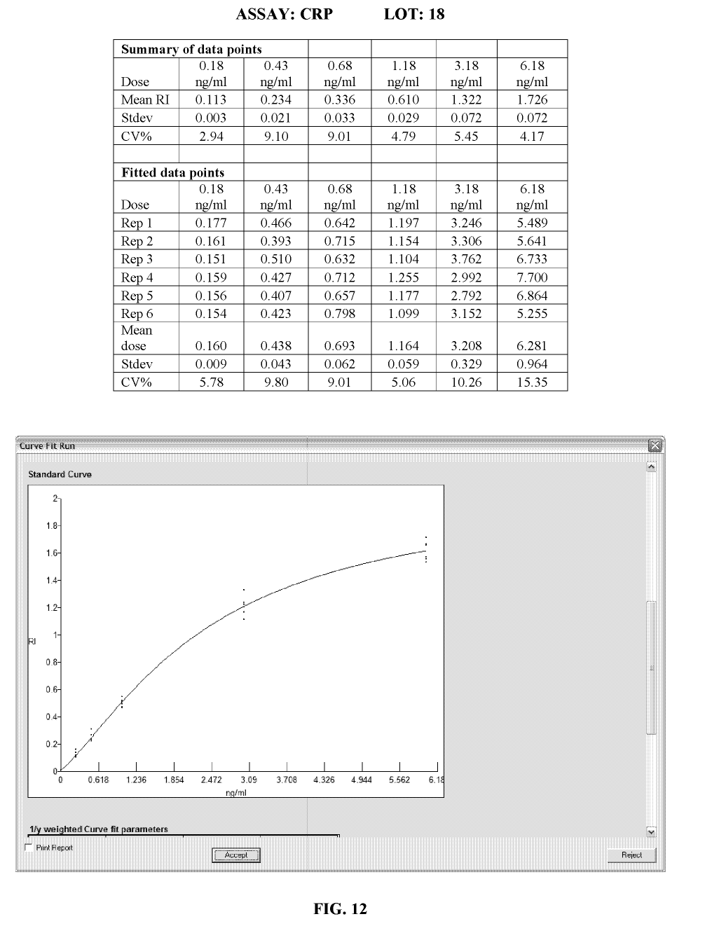

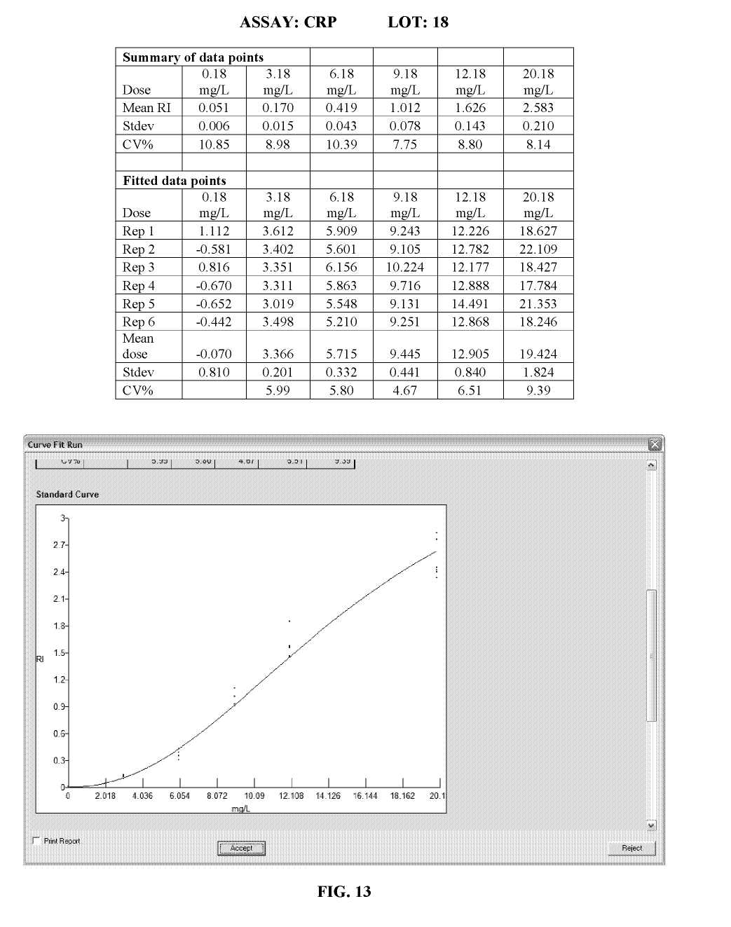

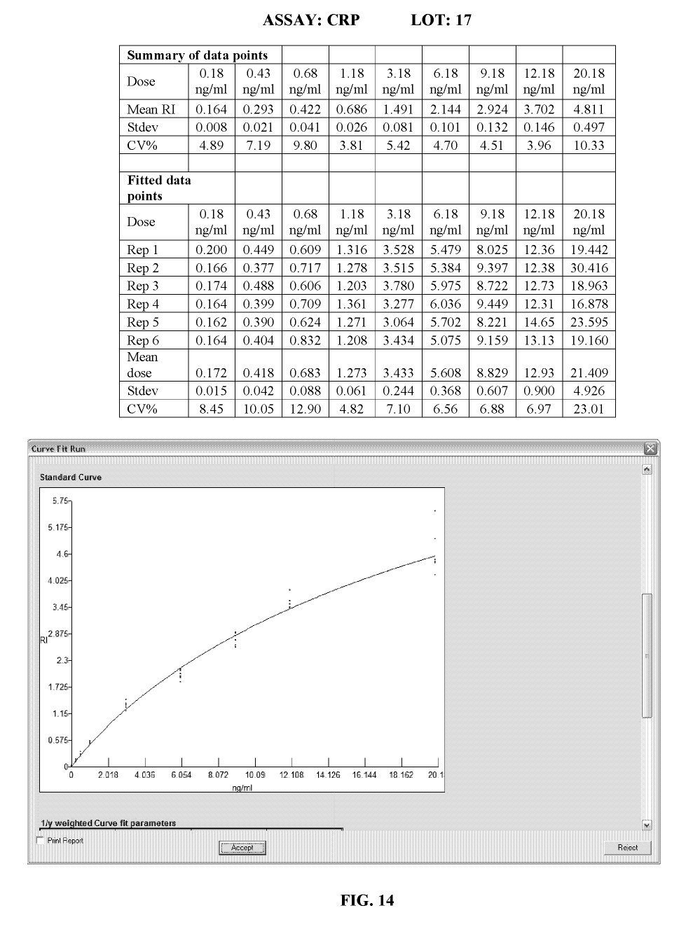

Results in FIGS. 12, 13 and 14 show that whereas the dynamic range of the RI versus CRP concentration from T1 was between approximately 0.2 and 3 mg/L CRP and the dynamic range of the RI from T2 versus CRP concentration was between approximately 3 and approximately 20 mg/L, combining the two standard curves gives a combined standard curve with a dynamic range between approximately 0.2 and 20 mg/L CRP. Thus, the lateral flow assay described in this example can accurately determine the CRP concentration in a patient sample between 0.2 and 20 mg/L without sample dilution.

### Example 2

### 3-Band CRP Assay

The strips were prepared exactly as described in Example 1 except for the following changes: (1) three test bands were coated on the nitrocellulose instead of two. T1 was coated with monoclonal anti-CRP 12D8 at a concentration of 0.2 mg/ml, T2 was coated with monoclonal anti-CRP 12D8 at a concentration of 0.5 mg/ml and T3 was coated with monoclonal anti-CRP 12D8 at a concentration of 1 mg/ml; (2) The high control (HC) was goat anti-mouse IgG coated at a concentration of 1 mg/ml; (3) The order of the bands in the test strip described in Thayer et al, U.S. Pat. No. 6,528,323 was (in order of distance from the sample application zone was: HC, T1, T2 and T3; (4) There was no low control; and (5) No BSA-DNP conjugate was coated on the conjugate pad.

Strips were run exactly as described in Example 1. RI was calculated by dividing the density of reflectance (DR) from either T1, T2, or T3 by the density of reflectance of the high control (HC).

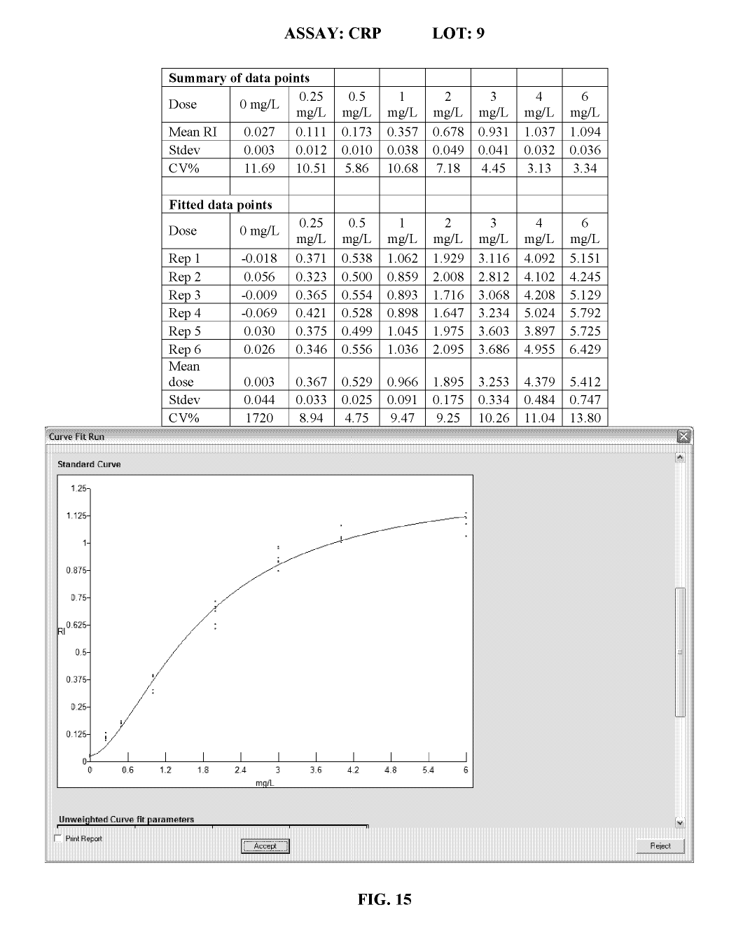

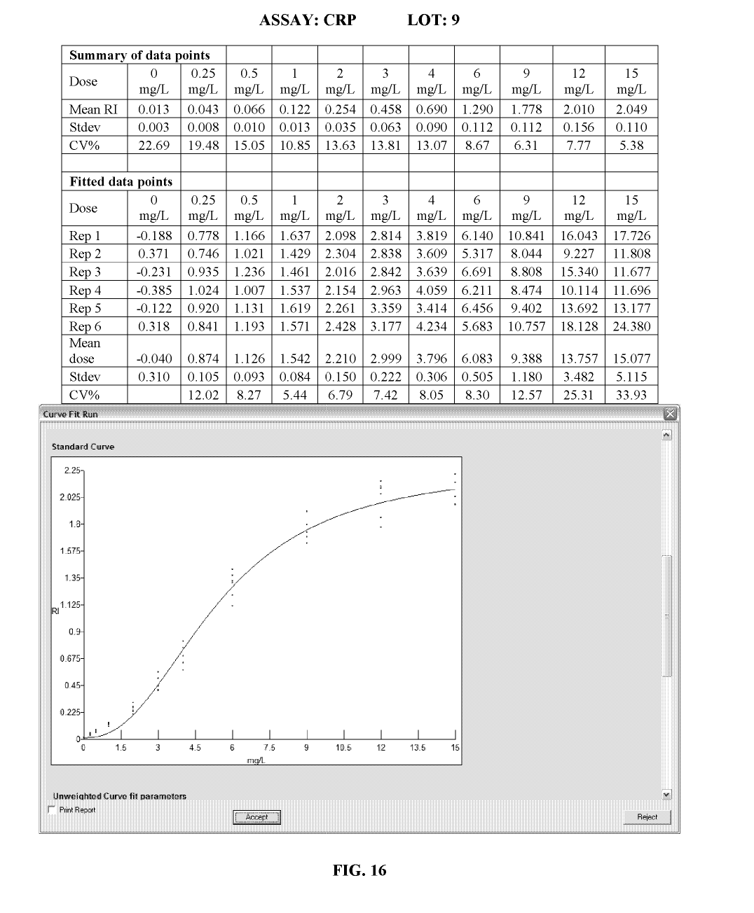

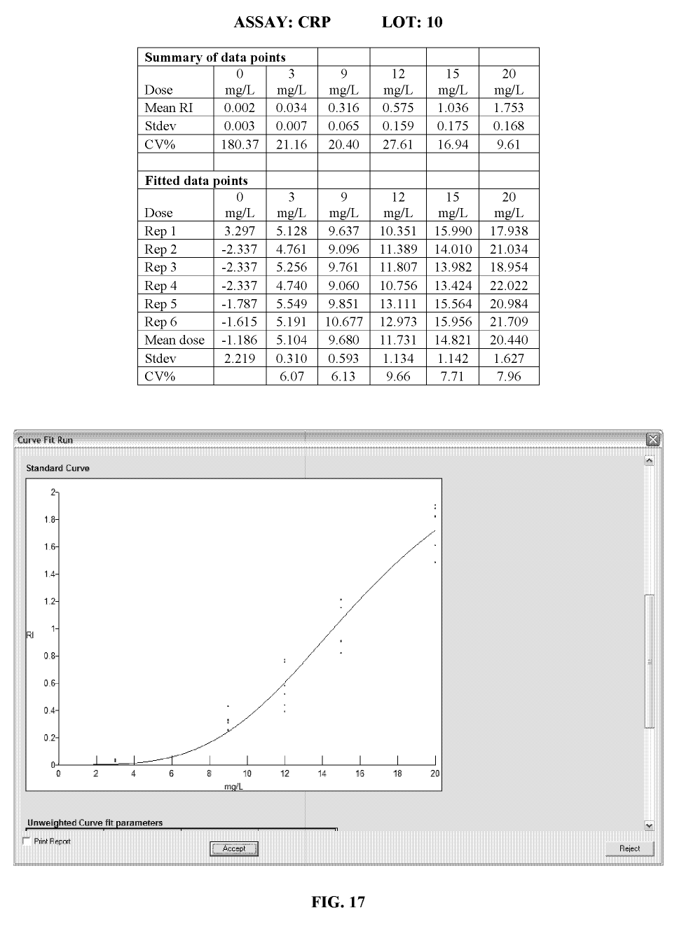

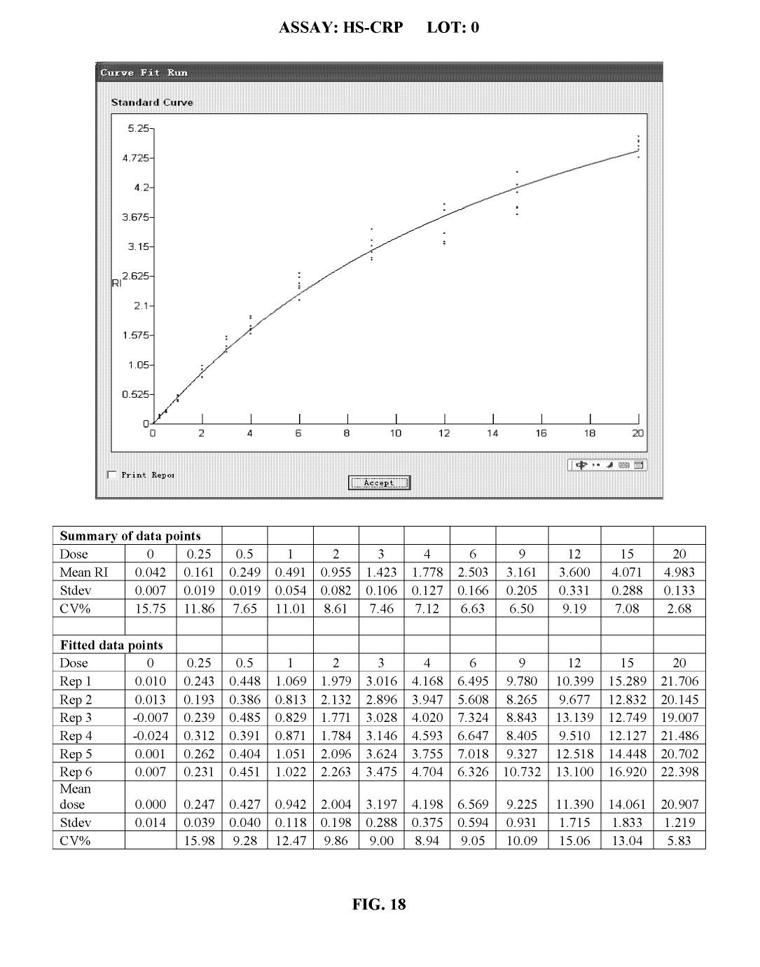

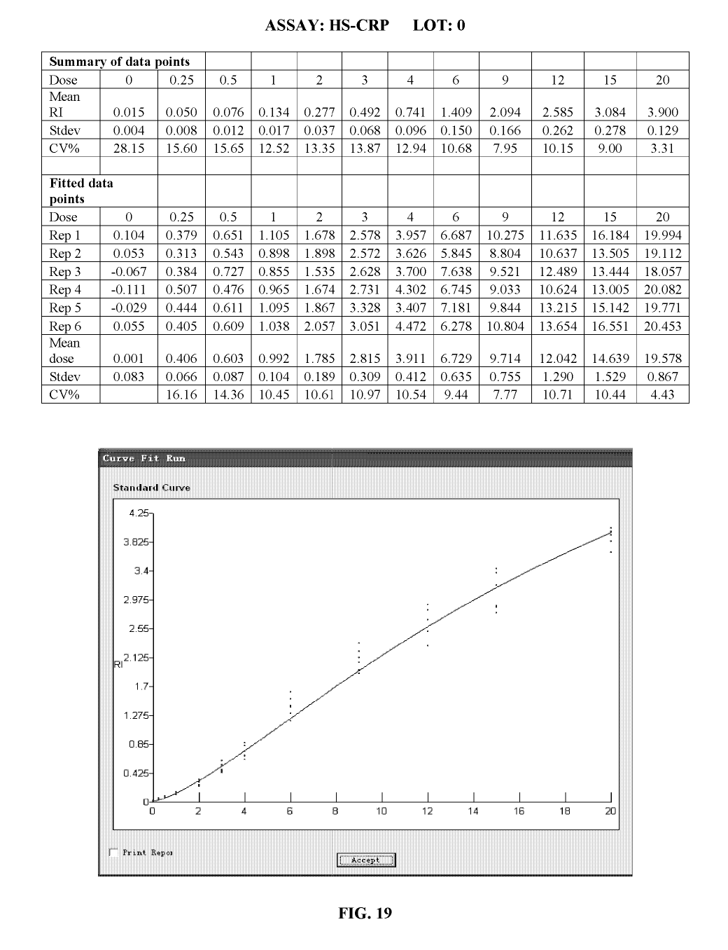

FIGS. 15, 16, and 17 demonstrate that T1 RI may be used to determine CRP concentrations between approximately 0.25 and approximately 3 mg/L, T2 RI may be used to determine CRP concentrations between approximately 2 and approximately 9 mg/L and T3 RI may be used to determine CRP concentrations between approximately 9 and approximately 20 mg/L. However, as shown in FIGS. 18 and 19, standard curves of the combined T2 and T3 RI values versus CRP concentration or the combined T1, T2 and T3 RI values versus CRP concentration may be used to extend the dynamic range and determine CRP concentrations between approximately 0.25 and 20 mg/L.

In addition to extending the dynamic range of lateral flow immunoassays assays using undiluted samples, three band CRP assays reveal other information about measurement of analyte levels in clinical samples. Although generalized inflammation is indicated by levels of 6 mg/L or higher of CRP, it is sometimes desirable to quantitatively determine higher concentrations of CRP in serum, plasma or whole blood. However, assays such as those described herein, which use undiluted patient samples, are subject to the high dose hook effect, also called a prozone. Although very high concentrations of CRP in patient samples (>20 mg/L) will not depress assay response below the cutoff level for generalized inflammation, inaccurate concentrations of CRP may be reported. However, as is shown in Table 1, for the three band assay described herein, as concentrations of CRP increase above 9 mg/L, the ratio of T3 DR/T1 DR increases rapidly, with the mean T3 DR/T1 DR ratio reaching 1.62 at 20 mg/L.

TABLE 1

| Standard (mg/L) | Mean T3/T1 |
|---|---|
| 6 | 0.092 |
| 9 | 0.249 |
| 12 | 0.542 |
| 15 | 0.953 |
| 20 | 1.608 |

Table 2 is a tabulation of mg/L measured from the combined standard curves of T2 and T3 from the 3-band CRP assay described above, mg/L CRP as determined by a reference assay and the T3 DR/T1 DR ratio from the three band CRP assay. These data demonstrate that whenever the reference assay and the 3-band CRP assay report CRP concentrations below 20 mg/L, the T3 DR/T1 DR ratio is greater than 1.6. However, whenever the concentration of CRP, as determined by the 3-band CRP assay, is less than 20 mg/L, and the concentration of CRP as determined by the reference assay is more than 20 mg/L, the ratio of T3 DR to T1 DR is less than approximately 1.6. Thus samples which give T3 DR/T1 DR of greater than 1.6 are samples where assay response in the 3-band CRP assay has been depressed by the high dose hook effect and contain more than 20 mg/L of CRP.

TABLE 2

| | 3 Band Assay | | Reference Assay |
|---|---|---|---|
| Sample | Result (mg/L) | T3/T1 | Result (mg/L) |
| 28 | 17.44 | 1.23 | 18.5 |
| 78 | 16.91 | 1.63 | 23 |
| 82 | 18.19 | 2.45 | 25.5 |
| 46 | >20 | 8.78 | 109.5 |
| 11 | 17.03 | 8.22 | 95 |
| 97 | >20 | 7.05 | 70.5 |
| 30 | 12.89 | 9.08 | 180.5 |
| 49 | 15.21 | 7.23 | 110 |
| 4 | 10.02 | 8.33 | 223 |

### Example 3

### Determination of NT-proBNP Concentration in a Sample

The test strips described in DiNello et al, U.S. Pat. No. 6,767,710 were prepared by coating Millipore HF 120 nitrocellulose (in order of distance from the buffer application zone) High control (HC): 1.0 mg/ml rabbit anti-DNP, Test band 1 (T1): 1.5 mg/ml monoclonal anti-NT-proBNP 15F11, Test band 2 (T2): 1.0 mg/ml monoclonal anti-NT-proBNP 15F11, and low control (LC): 0.5 mg/ml rabbit anti-DNP. Antibodies were dissolved in PBS, 5% trehalose, 5% methanol for coating and the nitrocellulose was coated using an IVEK flatbed striper at 1 μl/cm. Conjugates of monoclonal anti-NT-proBNP 5B6 and Monoclonal anti-NT-pro BNP 11D1 and 48 nm gold sol were prepared as described in Example 1 and the same antibodies were conjugated to 120 nm gold sol also as described in example 1. Conjugate pads were also prepared as described in example 1. The contribution to the OD 520 in a 1:20 dilution of the final coating solution from monoclonal anti-NT-proBNP 5B6-48 nm gold conjugate was 1.3, from monoclonal anti-proBNP 11D1-48 nm gold conjugate was 1.3, from monoclonal anti-NT-proBNP 5B6-120 nm gold conjugate was 0.156, from monoclonal anti-NT-proBNP 11D1-120 nm gold conjugate was 0.156 and from BSA-DNP-28 nm gold conjugate was: 0.39. Conjugate pads were dried overnight under vacuum and then treated for three days at 60° C.

For comparison purposes, nitrocellulose strips with only one test band were prepared, as above. Bands coated on these strips (in order of distance from the sample application zone) were: High control (HC): 1.0 mg/ml rabbit anti-DNP, Test band (T): 1 mg/ml monoclonal anti-NT-proBNP 15F11, and low control (LC): 0.5 mg/ml rabbit anti-DNP. Antibodies were dissolved in PBS, 5% trehalose, 5% methanol for coating and the nitrocellulose was coated using an IVEK flatbed striper at 1 μl/cm. Conjugates of monoclonal anti-NT-proBNP 5B6 and monoclonal anti-NT-pro BNP 11D1 and 120 nm gold sol and BSA-DNP-28 nm gold were prepared as described in Example 1. Conjugate pads were prepared as described in Example 1. The contribution to the OD 520 in a 1:20 dilution of the final coating solution from monoclonal anti-NT-proBNP 5B6-120 nm gold conjugate was 0.65, from monoclonal anti-proBNP 11D1-48 nm gold conjugate was 0.65 and from BSA-DNP-28 nm gold conjugate was 0.26.

Assays using the strips described above were carried out in a ReLIA 2 Instrument (ReLIA Diagnostic Systems, Burlingame, Calif.). The cassette was placed in the cassette tray of the instrument and sample specific information was entered. A 50 μl aliquot of prewetting buffer was then added to Port 1 of the cassette described in DiNello et al, U.S. Pat. No. 6,767,710. The addition of buffer was detected by a sensor and the cassette was withdrawn into the instrument for a countdown of 120 seconds. At the end of this time, the cassette tray was ejected from the instrument and the user was prompted to add 100 μl of a serum/plasma sample or 120 μl of a whole blood sample. Sample addition was also detected by a sensor initiating the test sequence. The assay was carried out under predefined assay conditions (20 min. at 33° C.). At the end of this time, the instrument determined the density of reflectance (DR) from each test and control band and the results were then accessed using the computer interfaced with the instrument.

The relative intensity (RI) of each band was calculated by dividing the density of reflectance (DR) from either T1 or T2 by the mean density of reflectance of the high control (HC) and low control (LC) bands.

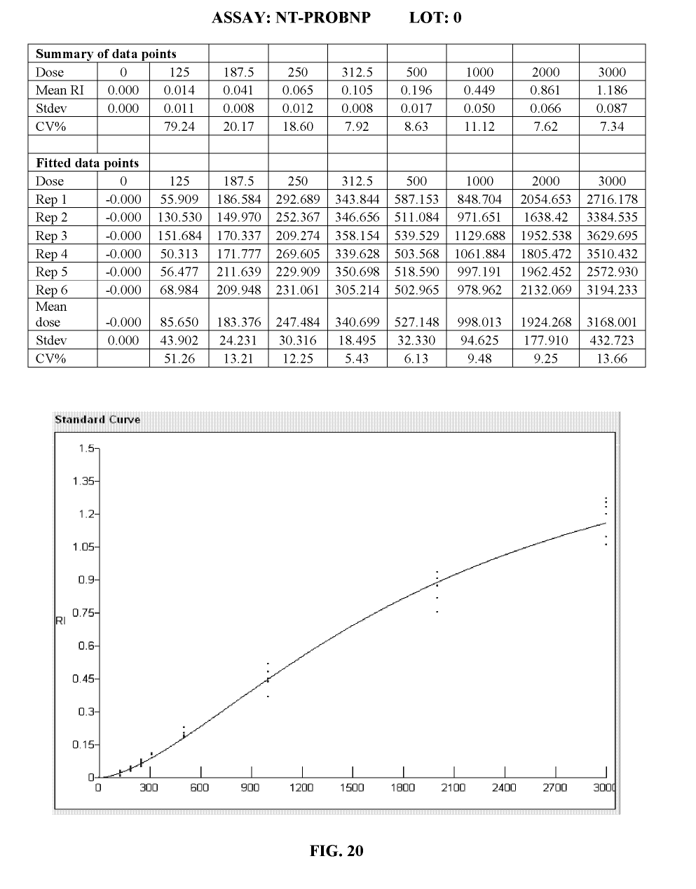

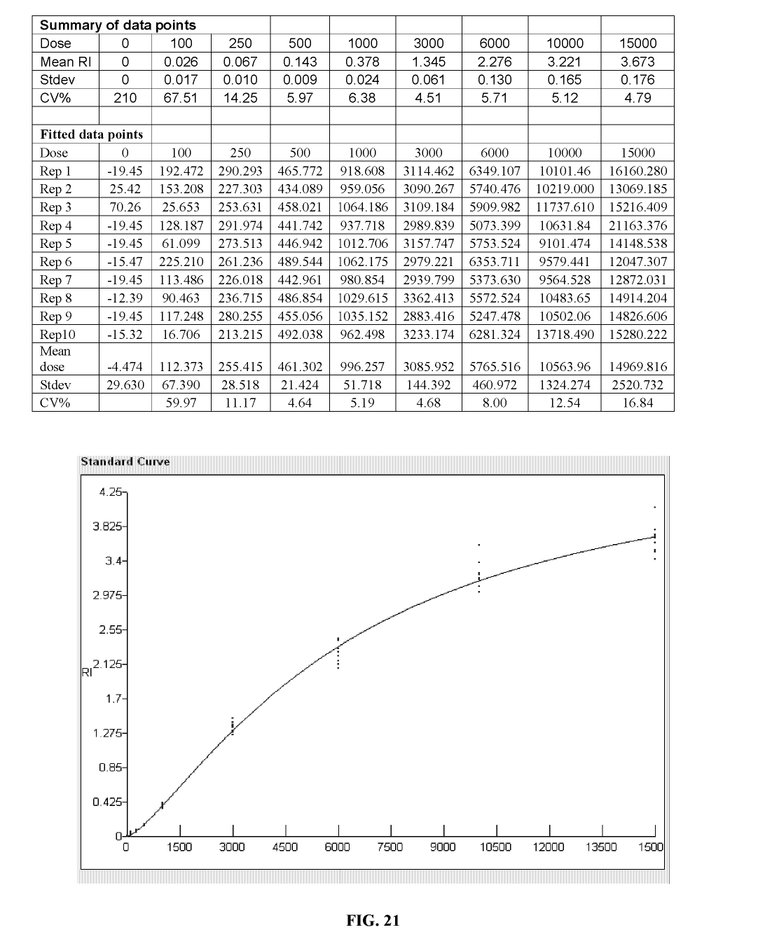

FIG. 20 demonstrates that the dynamic range of the one band NT-proBNP assay was between about 85.65 pg/ml and about 3000 pg/ml. Above 3000 pg/ml, the standard curve flattens to the extent that concentrations above 3000 pg/ml cannot be distinguished from 3000 pg/ml. However, FIG. 21 demonstrates that the two band assay, where the RI of T1 and the RI of T2 were combined by addition of the two individual RI values had a dynamic range which extends from about 88.89 pg/ml to greater than 10,000 pg/ml, enabling the user to accurately measure concentrations of above 3000 pg/ml without sample dilution.

All patents and publications referred to herein are expressly incorporated by reference in their entirety.

Although the invention has been described with reference to the presently preferred embodiments, it should be understood that various modifications can be made without departing from the spirit of the invention. Accordingly, the invention is limited only by the following claims.

What is claimed is:

1. A test strip comprising:
a chromatographic strip with a first end and a second end that comprises at least a first reaction region, and a second reaction region, wherein each reaction region comprises a capture agent that specifically binds the same analyte that may be present in a sample; and
an absorbent pad at the first end of the chromatographic strip,
wherein the test strip allows lateral flow of the sample such that as the sample is added to the chromatographic strip the sample can flow across each reaction region, thereby allowing the capture agent therein to bind at least a part of the analyte.

2. The test strip of claim 1, wherein the capture agent present in the second reaction region is the same as the capture agent present in the first reaction region.

3. The test strip of claim 1, wherein the capture agent present in the second reaction region is different from the capture agent present in the first reaction region.

4. The test strip of claim 1, wherein the capture agent present in the second reaction region is the same as the capture agent present in the first reaction region, and wherein concentration or quantity of the capture agent present in the first reaction region is the same as concentration or quantity of the capture agent present in the second reaction region.

5. The test strip of claim 1, wherein the capture agent present in the second reaction region is the same as the capture agent present in the first reaction region, and wherein concentration or quantity of the capture agent present in the first reaction region is different from concentration or quantity of the capture agent present in the second reaction region.

6. The test strip of claim 1, wherein the capture agent present in the second reaction region is the same as the capture agent present in the first reaction region, wherein the sample added to the test strip flows along the test strip towards the reaction regions until it reaches the first reaction region where a part of the analyte is captured by the capture agent, thereby depleting the sample of the analyte, wherein the sample continues to flow until it reaches the second reaction region where analyte, if remaining in the sample, is captured by the capture agent, and wherein the presence of the analyte in the sample is determined, and optionally, the quantity of the analyte measured.

7. The test strip of claim 3, wherein concentration or quantity of the capture agent present in the second reaction region is the same as concentration or quantity of the capture agent present in the first reaction region.

8. The test strip of claim 3, wherein concentration or quantity of the capture agent present in the second reaction region is different from concentration or quantity of the capture agent present in the first reaction region.

9. The test strip of claim 1, wherein the test strip supports unidirectional lateral flow.

10. The test strip of claim 1, wherein the test strip supports bidirectional lateral flow.

11. The test strip of claim 1, wherein the test strip further comprises a third reaction region, wherein the third reaction region comprises a capture agent that specifically binds the same analyte, wherein the sample added to the test strip flows along the test strip towards the reaction regions until it reaches the first reaction region where a part of the analyte is captured by the capture agent present in the first reaction region, wherein the sample continues to flow until it reaches the second reaction region where the analyte again binds the capture agent present in the second reaction region, wherein the sample continues to flow until it reaches the third reaction region where analyte, if remaining in the sample, is captured by the capture agent present in the third reaction region, and wherein the presence of the analyte in the sample is determined, and optionally, the quantity of the analyte measured.

12. The test strip of claim 1, wherein the test strip further comprises a third reaction region, and wherein the capture agent present in the second and third reaction regions are the same as the capture agent present in the first reaction region.

13. The test strip of claim 1, wherein the test strip further comprises a third reaction region, and wherein the capture agent present in the second or the third reaction region is different from the capture agent present in the first reaction region, but wherein each capture agent specifically binds the same analyte in the sample.

14. The test strip of claim 1, wherein the test strip further comprises a third, fourth, fifth, sixth, seventh or eighth reaction region, wherein the capture agent present in any reaction region is, but need not be, different from the capture agent present in any other reaction region, and wherein each capture agent specifically binds the same analyte in the sample.

15. The test strip of claim 1, wherein the test strip further comprises a sample addition zone to which sample is added, wherein sample when added, flows along the test strip from the sample addition zone to the first reaction region and then the second reaction region.

16. The test strip of claim 15, wherein the sample addition zone is located near the first end, at the second end, or near the second end of the chromatographic strip.

17. The test strip of claim 5, wherein the test strip further comprises a sample addition zone to which sample is added, wherein sample when added, flows along the test strip from the sample addition zone to the first reaction region and then the second reaction region and wherein concentration or quantity of the capture agent present in the first reaction region is higher than concentration or quantity of the capture agent present in the second reaction region.

18. The test strip of claim 5, wherein the test strip further comprises a sample addition zone to which sample is added, wherein sample when added, flows along the test strip from the sample addition zone to the first reaction region and then the second reaction region and wherein concentration or quantity of the capture agent present in the first reaction region is lower than concentration or quantity of the capture agent present in the second reaction region.

19. The test strip of claim 8, wherein the test strip further comprises a sample addition zone to which sample is added, wherein sample when added, flows along the test strip from the sample addition zone to the first reaction region and then the second reaction region and wherein concentration or quantity of the capture agent present in the first reaction region is higher than concentration or quantity of the capture agent present in the second reaction region.

20. The test strip of claim 8, wherein the test strip further comprises a sample addition zone to which sample is added, wherein sample when added, flows along the test strip from the sample addition zone to the first reaction region and then the second reaction region and wherein concentration or quantity of the capture agent present in the first reaction region is lower than concentration or quantity of the capture agent present in the second reaction region.

21. The test strip of claim 1, wherein the test strip is capable of determining the presence of the analyte when the analyte is present in the sample in concentrations ranging from about 0.0001 ng/ml to about 1 mg/ml.

22. The test strip of claim 15, wherein the test strip further comprises a conjugate region comprising a conjugate, wherein the conjugate region is located at the sample addition zone or between the sample addition zone and the first reaction region, and wherein sample when added, flows along the test strip from the sample addition zone to the conjugate region before reaching the first reaction region and the second reaction region.

23. The test strip of claim 22, wherein the test strip comprises a conjugate pad comprising the conjugate, wherein the conjugate pad is located in the conjugate region.

24. A test strip comprising:
a chromatographic strip that comprises at least two reaction regions, a first reaction region, and a second reaction region,
wherein each reaction region comprises a capture agent that specifically binds the same analyte that may be present in a sample, and
wherein the two or more reaction regions expand the dynamic range of the test strip.

25. The test strip of claim 24, wherein the test strip further comprises a third reaction region, wherein the third reaction region comprises a capture agent that specifically binds the same analyte.

26. The test strip of claim 24, wherein the dynamic range of the test strip is expanded at the lower end of concentration of the analyte.

27. The test strip of claim 24, wherein the dynamic range of the test strip is expanded at the higher end of concentration of the analyte.

28. The test strip of claim 24, wherein the dynamic range of the test strip is expanded at the lower and higher ends of concentration of the analyte.

29. A method for detecting the presence of an analyte in a sample comprising:
(a) delivering a sample to the test strip of claim 1,
(b) allowing the sample to flow along the test strip towards the reaction regions until it reaches the first reaction region and then the second reaction region;
(c) depleting the sample of analyte progressively by capturing at least a part of the analyte in the first reaction region, and if analyte remains in the sample, then in the second reaction region;
(d) determining the presence of the analyte in the sample based on intensity of a signal detected from the first reaction region, the second reaction region or a combination thereof; and optionally,
(e) measuring the quantity of, the analyte in the sample.

30. A method for detecting the presence of an analyte in a sample comprising:
(a) delivering a sample to the test strip of claim 24,
(b) allowing the sample to flow along the test strip towards the reaction regions until it reaches the first reaction region and then the second reaction region;
(c) depleting the sample of analyte progressively by capturing at least a part of the analyte in the first reaction region, and if analyte remains in the sample, then in the second reaction region;
(d) determining the presence of the analyte in the sample based on intensity of a signal detected from the first reaction region, the second reaction region or a combination thereof; and optionally,
(e) measuring the quantity of, the analyte in the sample.

31. The method of claim 29, wherein the test strip further comprises a third reaction region, that comprises a capture agent that specifically binds the same analyte in the sample, wherein the presence of the analyte in the sample is determined based on intensity of a signal detected from the first reaction region, the second reaction region, the third reaction region, or any combination thereof

32. The method of claim 30, wherein the test strip further comprises a third reaction region, that comprises a capture agent that specifically binds the same analyte in the sample, wherein the presence of the analyte in the sample is determined based on intensity of a signal detected from the first reaction region, the second reaction region, the third reaction region, or any combination thereof.

33. The method of claim 29, wherein the presence of the analyte in the sample is determined, or the quantity of the analyte in the sample is measured based on intensity of a signal from the first reaction region only.

34. The method of claim 29, wherein the presence of the analyte in the sample is determined, or the quantity of the analyte in the sample is measured based on intensity of a signal from the second reaction region only.

35. The method of claim 29, wherein the presence of the analyte in the sample is determined, or the quantity of the analyte in the sample is measured based on a combination of signal intensities from the first reaction region and the second reaction region.

36. The method of claim 31, wherein the presence of the analyte in the sample is determined, or the quantity of the analyte in the sample is measured based on a combination of signal intensities from the first reaction region, the second reaction region and the third reaction region.

37. The method of claim 29, wherein the signal is a reflectance, fluorescence, luminescence, radiation, or a radiofrequency signal.
